(19) United States
(12) Patent Application Publication
(10) Pub. No.: US 2024/0378437 A1
(43) Pub. Date: Nov. 14, 2024

(54) ANALYZING AND SELECTING PREDICTIVE ELECTROCARDIOGRAM FEATURES

(71) Applicants: Mayo Foundation for Medical Education and Research, Rochester, MN (US); Regents of the University of Minnesota, Minneapolis, MN (US)

(72) Inventors: Paul A. Friedman, Rochester, MN (US); Itzhak Zachi Attia, Rochester, MN (US); Gilad Lerman, St. Paul, MN (US)

(21) Appl. No.: 18/563,316

(22) PCT Filed: May 23, 2022
(86) PCT No.: PCT/US2022/030476

§ 371 Date: Nov. 21, 2023

Related U.S. Application Data
(60) Provisional application No. 63/191,920, filed on May 21, 2021.

(51) Int. Cl. G06N 3/08; A61B 5/319; A61B 5/35

(52) U.S. Cl. CPC G06N 3/08; A61B 5/319; A61B 5/35

(57) ABSTRACT

In some aspects, values of features obtained from training first and second machine-learning models are analyzed to correlate at least a subset of features from the first machine-learning model with at least a subset of features from the second machine-learning model. The correlated features are then applied to update the first or second machine-learning model, or to train a third machine-learning model. In other aspects, a generator machine-learning model processes a seed and a target characteristic indicator to generate a synthetic ECG signal. The synthetic ECG signal is biased according to a target physiological characteristic represented by the target characteristic indicator. The generator machine-learning model can be trained using an expert machine-learning model and in an adversarial process with a discriminator machine-learning model.

### CROSS-REFERENCE TO RELATED APPLICATIONS

This application claims priority to U.S. Application Ser. No. 63/191,920, filed May 21, 2021, the entire contents of which are incorporated by reference into the disclosure of the present application.

### BACKGROUND

This specification relates to machine-learning techniques, particularly as applied to electrocardiograms or other measurements of electrical activity in a mammal (e.g., electroencephalograms).

Neural networks are machine-learning models that employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters.

Machine learning models such as convolutional neural networks enable computers to develop data-derived rules to solve complex classification problems without human knowledge regarding the structure of the input. For example, neural networks have been trained to analyze inputs representative of electrocardiograms (ECGs) of a person or other mammal, and to predict from the ECG conditions such as arrhythmias based on features that may not be apparent from human inspection of the ECG. Some models are configured to generate predictions based on complete representations of an ECG (e.g., time-indexed values of an ECG signal over one or more beats). Other models process inputs representing derived features of an ECG signal such as characteristics of the QRS-complex or T-wave.

### SUMMARY

In a first aspect, implementations include computer-implemented methods for correlating features from machine-learning models. First values for a first set of features from a first machine-learning model are obtained, where the first values for the first set of features were determined through a process of training the first machine-learning model to perform a particular classification task based on inputs that represent a signal. Second values for a second set of features from a second machine-learning model are obtained, where the second values for the second set of features are determined through a process of training the second machine-learning model to perform the particular classification task based on inputs that represent morphological features of the signal. The first and second values are processed to correlate at least a subset of the first set of features with at least a subset of the second set of features. The correlated features can then be used to update the first machine-learning model, update the second machine-learning model, or train another machine-learning model.

These and other implementations can further include one or more of the following features.

The first machine-learning model and the second machine-learning model can be neural networks.

The signal can be an electrocardiogram (ECG) or an electroencephalogram (EEG).

The morphological features of the signal can include human-selected features, where the first set of features includes features that are not human-selected features.

The first set of features can correspond to a last hidden layer of a neural network.

The correlation can be used to update the second machine-learning model by reducing the second set of features, or the correlation can be used to update the first machine-learning model by reducing the first set of features.

In a second aspect, implementations include methods for training a computer-implemented system to generate synthetic electrocardiogram (ECG) signals. The methods can include obtaining a seed and a target characteristic indicator, where the target characteristic indicator represents a target physiological characteristic for a patient (e.g., a fictional patient); processing, with a generator machine-learning model, the seed and the target characteristic indicator to generate a synthetic ECG signal; processing, with an expert machine-learning model, the synthetic ECG signal to generate a patient characteristic prediction; processing, with a discriminator machine-learning model, the synthetic ECG signal to generate an authenticity prediction; determining a generator loss based on (i) a first comparison of the patient characteristic prediction to the target characteristic indicator and (ii) a second comparison of the authenticity prediction an authenticity indicator that indicates the synthetic ECG signal was inauthentic; and updating parameters of the generator machine-learning model based on the generator loss.

These and other implementations can further include one or more of the following features.

The target physiological characteristic of the patient can be a sex of the patient, an age of the patient, or a ventricular function of the patient. The ventricular function of the patient can include an ejection fraction, a heart rate, an arrhythmia, or a left ventricular dysfunction.

The target characteristic indicator represents the target physiological characteristic for the patient on a continuous, non-binary scale.

The expert machine-learning model can be pre-trained to generate patient characteristic predictions based on ECG signals, the patient characteristic prediction comprising sex, age, or ventricular function.

The seed can be a randomly selected value within a range of values.

The generator machine-learning model can include a first convolutional neural network and the discriminator machine-learning model comprises a second convolutional neural network.

The generator machine-learning model and the discriminator machine-learning model can be alternately trained in respective epochs that involve processing one or more training samples in each epoch. Parameters of the discriminator machine-learning model can be held constant while training the generator machine-learning model, and the parameters of the generator machine-learning model can be held constant while training the discriminator machine-learning model.

Updating the parameters of the generator machine-learning model based on the generator loss can include back-propagating the generator loss through the discriminator machine-learning model, the expert machine-learning model, and the generator machine-learning model, and using gradients from the back-propagation to update the parameters of the generator machine-learning model.

Determining the generator loss can include weighting the first comparison greater than the second comparison. Alternatively, determining the generator loss can include weighting the first comparison less than the second comparison.

In a third aspect, implementations include methods for generating a synthetic electrocardiogram (ECG) signal. The method can include operations of obtaining a seed and a target characteristic indicator, wherein the target characteristic indicator represents a target physiological characteristic for a patient; and processing, with a generator machine-learning model, the seed and the target characteristic indicator to generate the synthetic ECG signal, wherein the generator machine-learning model biases the synthetic ECG signal according to the target physiological characteristic represented by the target characteristic indicator.

In a fourth aspect, implementations include a training system comprising a generator machine-learning model implemented on one or more processors; a discriminator machine-learning model implemented on one or more processors; and an expert machine-learning model implemented on one or more processors; wherein one or more processors of the training system are configured to perform operations comprising: obtaining a seed and a target characteristic indicator, wherein the target characteristic indicator represents a target physiological characteristic for a patient; processing, with the generator machine-learning model, the seed and the target characteristic indicator to generate a synthetic ECG signal; processing, with the expert machine-learning model, the synthetic ECG signal to generate a patient characteristic prediction; processing, with the discriminator machine-learning model, the synthetic ECG signal to generate an authenticity prediction; determining a generator loss based on (i) a comparison of the patient characteristic prediction to the target characteristic indicator and (ii) a comparison of the authenticity prediction an authenticity indicator that indicates the synthetic ECG signal was inauthentic; and updating parameters of the generator machine-learning model based on the generator loss.

The techniques disclosed in this specification can achieve specific advantages in particular implementations. For example, synthesizing ECG signals that are biased according to targeted physiological characteristics, may allow researchers and clinicians to better understand expert models by replacing limited datasets used for explainability with synthetic ones that are potentially infinite in size and quality and that are created specifically for the model in need of explanation Since the generator can be trained unlabeled data and expert model, the disclosed systems can explain expert models trained on small labeled datasets. Moreover, since ECGs with specific network-predicted characteristics are generated, these can be visually inspected to identify feature changes that drive classification (for example, what changes in an ECG as changes from being read as female to male), or statistical analysis can be applied to various features to further explore them (e.g., what happens to T-wave peak as the ECG sex changes from male to female). This understanding may add robustness against adversarial attacks, by underscoring the features that drive classification.

The ECG synthesizers disclosed herein can also be used to research target model bias and fairness. A generator model can be trained, for example, that is the product of multiple expert models' labels. If one AI-ECG model was trained to detect low ejection fraction (EF) using a dataset containing only Caucasian patients, and a second AI-ECG model is designed to determine whether a person is Caucasian or African American from ECG that was trained on patients without LVD labels, an EGAN can combine information from both models and synthesize ECGs that have both race and EF information. This allows generation of a spectrum of synthetic ECGs from African American patients with which to assess the AI-ECG LVD model to assess whether it performs differently based on race.

Synthesizing ECG signals can also have important implications for privacy, especially since an authentic ECG is a uniquely identifying fingerprint of a patient. Synthetic ECGs can be used that lack specific patient identity information while preserving the physiological features of interest (such as age, sex, ventricular function, or any other characteristic detectable by an AI-ECG expert model).

Additional features and advantages will be appreciated and recognized by persons of ordinary skill in the art in light of the following descriptions, the figures, and the claims.

### DESCRIPTION OF DRAWINGS

FIG. 1 depicts an example cardiac anatomy and an electrocardiogram (ECG) signal. The heart has four chambers, the upper chambers, the atria are activated by the signal reflected in the ECG as the P wave; the lower chambers, the ventricles are rapidly activated resulting in the QRS complex, and the relaxation of the ventricles (repolarization) is represented by the smoother T wave. A number of human-selected features, such as the peak amplitude of the various waves, the areas and widths of the different waves, deviation from baseline and other morphological characteristics have a known biological mechanism and associations with specific pathologies.

FIG. 2 depicts various classifier architectures configured to use human-selected or neural-network selected features.

FIG. 3 depict plots demonstrating canonical correlation between human-selected and neural network-selected features.

FIG. 4 depicts a plot demonstrating a proportion of residual variance explained as a function of principal components.

FIG. 5 depicts an estimate of human-selection features using neural network-selected features.

FIG. 6 depicts a table of R² statistics as a measure of variance explainability for human features in the two networks (sex or age). Human features that were extracted from each lead separately had very similar R-squared among the leads; the R-squared value is presented for the lead with the highest value. Features that were derived from all 12 leads together are present as is. The features are sorted based on the R-squared value from the highest score to the lowest.

FIG. 7 depicts a system implemented according to a master-student structure.

FIG. 8 depicts an explanatory generative adversarial network system for training a generator machine-learning model to produce realistic and physiologically-biased synthetic ECG signals.

FIG. 9 depicts a flowchart of an example process for generating a physiologically biased synthetic ECG signal.

FIGS. 10A-10B depict flowcharts of an example process for training the explanatory generative adversarial network for physiologically-biased ECG signal creation.

FIG. 11 depicts an architecture of an example explanatory GAN.

### DETAILED DESCRIPTION

Machine-learning models have been developed for tasks such as detecting asymptomatic left ventricular dysfunction from an electrocardiogram (ECG), and determining age, sex and cardiovascular risk from fundus photography. Network structures used to identify the presence of life-threatening diseases from an ECG can also be used to determine whether a person is male or female from a given ECG, depending on how the network is trained. To distinguish these tasks, during model training, the ground truth labels represent the specific characteristic that the network is to learn. In a convolutional neural network, instead of using human-selected features for signal processing, network features are created by projecting the input on a set of weights, and optimizing the weights in a nonlinear manner using labels during the training phase, with the objective of lowering the overall estimation or classification error. Through an iterative process, the network learns relevant rules and applies them to extract pertinent features for the specific test it is trained to solve. Because deep learning can replace human-engineered, hardcoded rules with computer-generated dynamically created rules based on data, biases in feature selection are possibly removed and human limitations can be overcome. However, deep learning is currently unexplainable.

This specification describes techniques that allow for understanding the features selected by neural networks for the analysis of the ECG. The ECG is the recording of the heart's electrical activity at a distance, i.e. from the body's surface. The ECG signal results from the activation of myocytes during different phases of the cardiac cycle. Since its discovery, the ECG has been used to record a number of physiologic and pathologic conditions, and with research and physician experience, the presence of specific features on the ECG tracing have been used to designate the presence or absence of specific biological conditions and disease states.

### Example 1—Neural Network Explainability

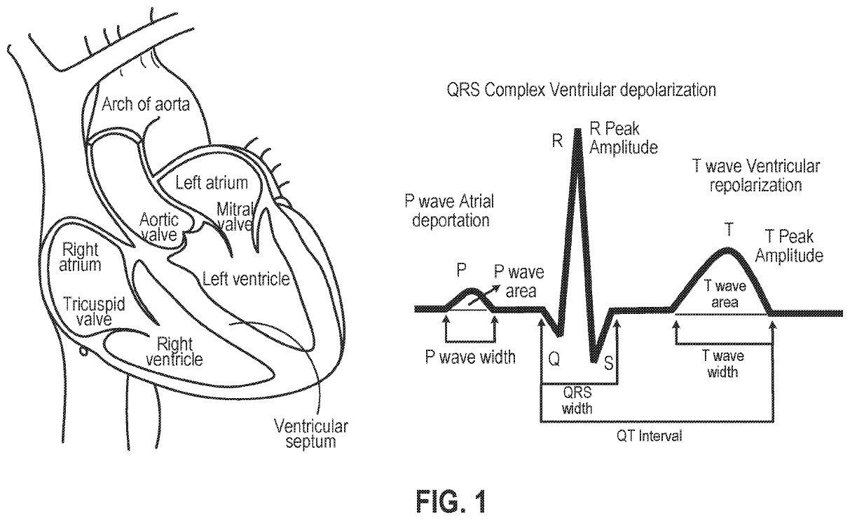

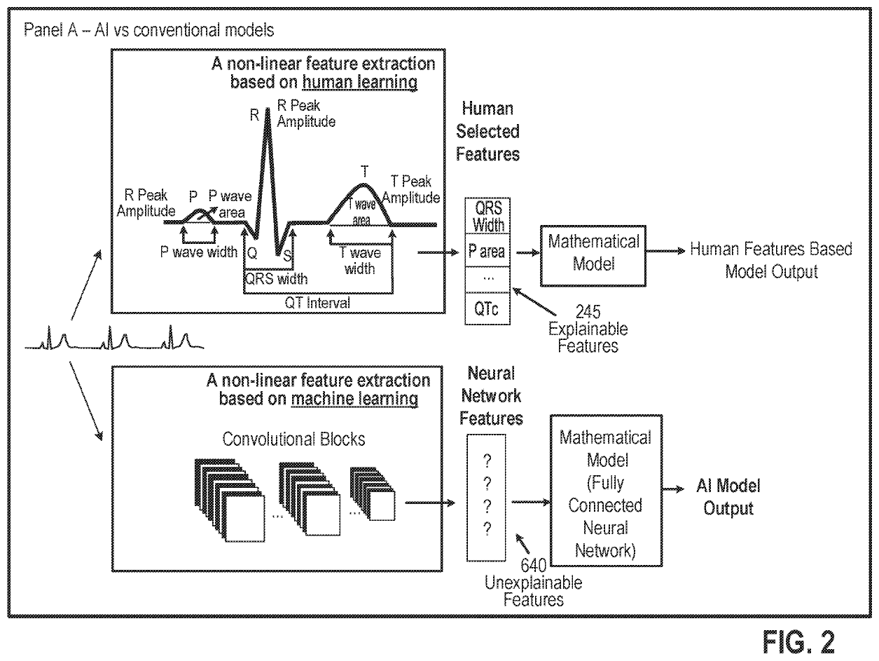

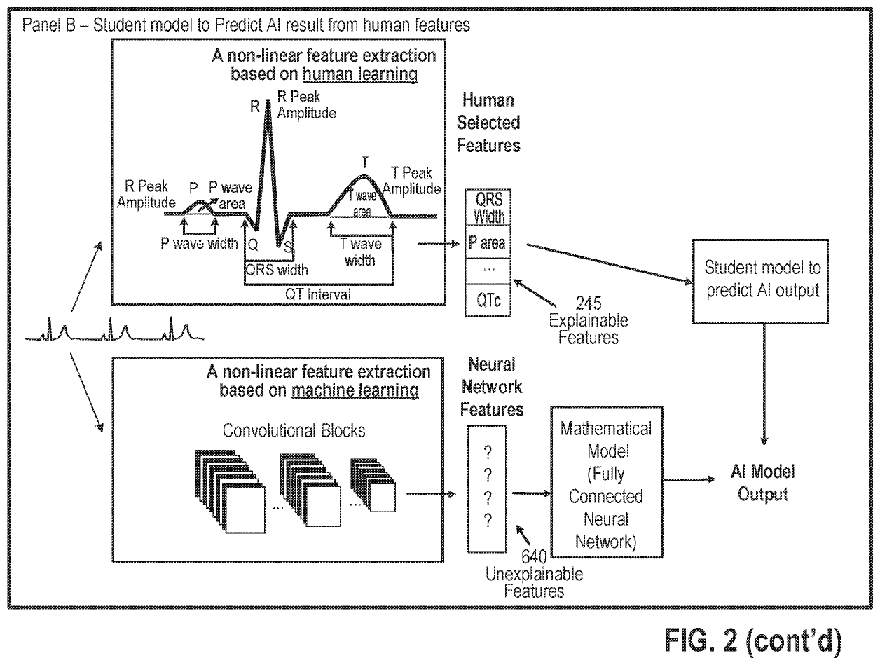

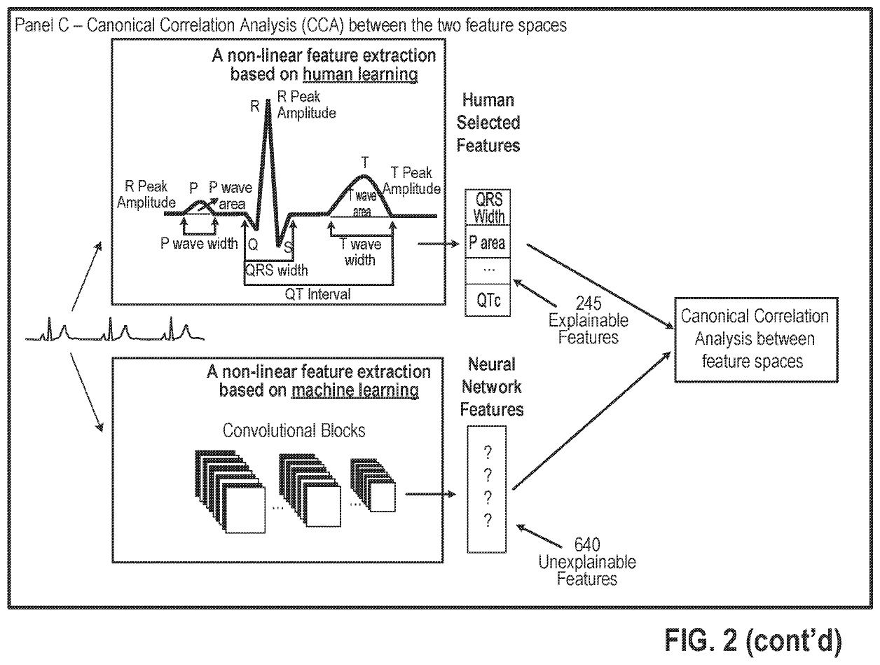

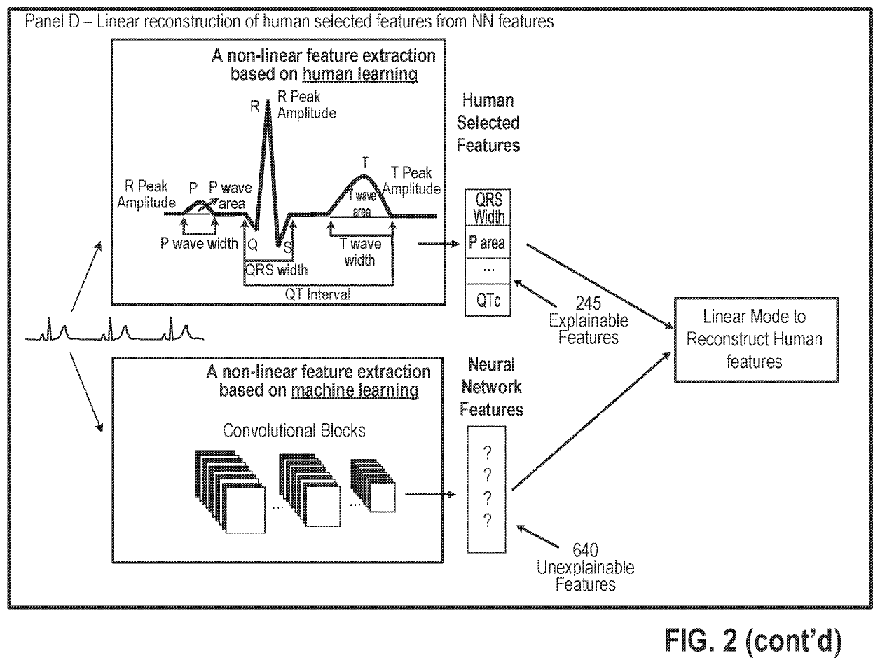

This example includes techniques from a study that references ECG features (such as ST segment elevation and T-wave amplitude) as the “vocabulary” for signal components fed into the model (e.g., the information the model uses to create its output), where the level of explanation depends on the volume and variety of features in the vocabulary. Some features are demonstrated in FIG. 1. It is recognized that multiple medical conditions may affect any individual feature, and any individual condition usually impacts multiple features. For diagnosis, clinicians are trained to recognize the most salient features associated with a given condition, while other changes, due to their small magnitude or variability are ignored. Human-crafted models weigh selected features to classify the absence or presence of a disease state, such as acute myocardial infarction, associated with the features of ST segment elevation. A neural network trained to detect the same condition from the same set of ECGs may or may not use similar signal features (FIG. 2A).

In this study, it was hypothesized that convolutional neural networks would extract similar, linearly correlated signal features, to those identified by humans. The study further hypothesized that the human-recognizable features can be used to explain to some extent the output of the neural networks and that the ability to explain the model will improve when using these human-recognizable features in a nonlinear way. To test these hypotheses, the study developed methods to extract neural network features and applied quantitative methods to measure the correlations between these features and the human extracted features. The study also explained the output of the neural networks by using student models. These methods help determine the explainability of the neural network by human-selected features and whether the network may find novel features that are not identified by humans.

The role of vocabulary and human explanation. The study defined a reasonable explanation as the translation of the rules used by a model for output determination to a language that a human expert can understand and replicate. These rules are specific to the problem one tries to solve. In order to define an explainable model for understanding neural networks (NN) for ECG processing, the study identified the domain specific vocabulary of human-selected features and basic methods for explainability and correlation. FIG. 2 provides a conceptual diagram of the proposed scheme.

ECG: background and structure. The electrocardiogram is the recording of the heart's electrical activity from the body's surface. Each individual myocyte has a resting negative electrical potential relative to the outside of the cell membrane due to the distribution of ions across it. Highly regulated voltage changes, controlled by membrane ion-channels, permit individual myocytes to depolarize, allowing electrical signals to propagate across the myocardial syncytium, which through electrical-mechanical coupling result in coordinated mechanical contraction. Each myocyte then repolarizes (recovers its resting negative potential) in preparation for the impulse to follow. The ECG is the summation in space and time of all of the individual myocyte voltage changes, and depicts the progression of electrical activation through the cardiac chambers (FIG. 1). Since the progression of cardiac wave fronts occur in three-dimensional space, the recording acquired from any given skin electrode will reflect the projection of the electrical vector at that particular point in space, so that a given signal will have a different appearance when recorded from different sites. Conversely, recording from multiple surface locations permits characterization of the cardiac site or origin of a given impulse. In the conventional ECG, 12 leads are recorded. The electrical activity in each heartbeat is divided into 5 main temporal waves (features), the P, Q, R, S and T waves (FIG. 1). The P wave represents atrial depolarization, the Q, R and S waves (typically referred to as the QRS complex) represent ventricular depolarization, and the T wave reflects ventricular repolarization.

Human-selected, explainable ECG features. When the ECG is acquired during normal rhythm, the morphology of each complex tends to have substantial homology among beats, so that an averaged beat can be used for morphologic feature extraction. The human-engineered process of feature extraction from ECG is non-trivial and nonlinear. It entails selection of specific signal components (e.g., the ST segment) which is useful if associated with specific conditions. For the present study, we used the human-defined features extracted and stored by the MUSE system. The system begins with the detection of each QRS complex in a segment and selection of a window of time around it, aligning the windows using a fiducial point in the QRS and averaging the complexes to a single representative beat. The features (FIG. 1) are extracted by finding the onset and offset of each component and identifying human-selected characteristics such as areas, maximum amplitudes, slopes, durations and so on for each constitutive element, creating a descriptive vocabulary for signal characteristics. The Muse system that the study use includes a matrix of human-selected features that are automatically extracted from each lead in a 12 lead ECG.

Experimental setting. The study used two previously described deep convolutional neural networks (NN), which were trained to classify ECGs for two different tasks: classification of sex and estimation of age. Using these networks, the study conducted experiments with 100,000 ECG signals from the Mayo Clinic digital data vault collected between January 1994 and February 2017 with institutional review board approval. ECGs were randomly selected from all-comers including cardiac and non-cardiac patients; 57.4% were male and the mean age was 58.7±15.7 years. The cohort used for this experiment was selected in a similar way to the ones used to train the original models the study sought to explain; however, the ECGs were independent from the ones used for the original training and validation of the networks.

Among the 100,000 ECG signals, 50,000 were used to train the student models (denoted as the student model training set) and 50,000 were used to evaluate the student models (denoted as the student model testing set). When training the original age and sex models, each ECG signal was zero padded from 5000×12 (10 seconds sampled at 500 Hz) to 5120×12, that is, for each of the 12 leads, the padded signal length was 5120 and no additional inputs were used. For the sex classification problem, labels of patient sex were provided as binary variables (0/1 for female/male) and the predicted output for the testing data obtained values in [0,1] indicating the probability of being a male. For the age estimation problem, labels of patient ages between 18 and 100 were provided and the predicted output for the testing data obtained values in [18,100].

The architecture of the age convolutional NN and the sex convolutional NN was the same except for the final output layer's activation (linear for age regression and SoftMax [binary classification] for sex). In both networks, the first component is composed of convolutional blocks, which reduce the dimension of each 5120×12 signal to 640. This was the feature extraction component of the network (FIG. 2). The study thus defined the NN selected features as the 640 outputs of the last convolutional layer. The next network component was the mathematical model, in this case fully connected layers that received the 640 features selected by the convolutional layers and manipulated them to obtain the desired output (sex classification or age estimation, FIG. 2A, bottom). Additionally, a total of 245 human-selected features derived from the median beat of each of the 100,000 ECGs was extracted using the Muse database (FIG. 2A, top). Some of the features were based on the morphology of a single lead and were extracted for each lead separately, but others, such as intervals (QT, RR, QRS) were calculated based on all 12 leads [20].

The study used the following notation, where for brevity, the study did not distinguish between sex classification and age estimation, as their models are identical except for the final output layer's activation:
Xₜᵣₐᵢₙ, Xₜₑₛₜ[N×640] were the student model training and testing matrices of NN features;
Zₜᵣₐᵢₙ, Zₜₑₛₜ[N×245] were the student model training and testing matrices of human-selected features;
yₜᵣₐᵢₙ, yₜₑₛₜ[N×1] were the student model training and testing output of the NN with the trained parameters.
The study used the NN outputs to train and test the student model and not the given labels since the study sought to explain the neural network output rather than create human features-based models.
Defining a student model and an explainability score. The study used a secondary student model designed to predict the output of the neural network using the human-selected features to explain the neural network. For simplicity, the study first considered a linear regression model. That is, the study defined a 245×1 vector w and a real number b and fit a standard least-squares linear regression model yₜᵣₐᵢₙ=Zₜᵣₐᵢₙw+b1_{N×1}, where 1_{N×1} is an N×1 vector of ones. The corresponding R² statistic, which incorporated the testing data, was interpreted as the linear explainability score. It has values between 0 and 1, where 1 designates perfect linear explanation and 0 an irrelevant vocabulary for linear explanation. It was computed as follows

$${R}^{2} = 1 - {‖ {y}_{test} - ( {Z}_{test} w + b {1}_{N \times 1} ) ‖}^{2} / {‖ {y}_{test} - \overline{{y}_{test}} {1}_{N \times 1} ‖}^{2} ,$$

where for a vector a, ā and ∥a∥ denote the mean and Euclidean norms, respectively.

The study also used a nonlinear model to explain the output using the human-selected features. This model used a fully connected network with two layers of 128 and 64 neurons and ReLU activation functions, followed by linear regression. The model was trained using a small set of hyperparameters and internally validated on a subset of the training data. Using matrices of parameters W_{245×128} and V_{128×64}, a vector w of size 64×1 and a scalar b, the nonlinear model was expressed as yₜᵣₐᵢₙ=f(Zₜᵣₐᵢₙ)=ReLU (ReLU (Z W_{245×128})V_{128×64}) w+b1_{N×1}. The study use the following R² statistic as the nonlinear explainability score:

$${R}^{2} = 1 - {‖ {y}_{test} - f ( {Z}_{test} ) ‖}^{2} / {‖ {y}_{test} - \overline{{y}_{test}} {1}_{N \times 1} ‖}^{2} .$$

The difference between the nonlinear and linear explainability scores quantified the improved performance of a nonlinear versus a linear model (FIG. 2B).

Canonical correlation between the feature spaces. The study used canonical correlation analysis (CCA) to assess the overall correlation between the spaces of the human-selected and NN features. CCA searches for linear transformations of the two sets of variables that maximize the cross correlation between the transformed sets. In our case, the study aimed to quantify the correlation between the rows of the N×640 and N×245 matrices Xₜₑₛₜ and Zₜₑₛₜ that represent NN and human-selected features respectively and the study pursued CCA as follows. The study first subtracted from each row of each matrix the mean of all rows of that matrix, so the variables were centered. For d≤min(rank(Xₜₑₛₜ), rank(Zₜₑₛₜ)), the study sought matrices T₁ and T₂ of coefficients of linear transformations, with respective sizes 640×d and 245×d, such that XₜₑₛₜT₁ and ZₜₑₛₜT₂ maximize the Frobenius norm of their cross correlation matrix. The singular values of this maximal cross correlation matrix are the canonical correlation coefficients. The study computed them as follows. Let U₁ and U₂ be the N×d matrices of left singular column vectors (arranged by descending order of singular values) of XₜₑₛₜT₁ and ZₜₑₛₜT₂, respectively. Then the canonical correlation coefficients are the singular values of the matrix U₁^{T}U₂. These numbers are between zero and 1, where higher numbers indicate higher correlation. Due to redundancies, one expects that many of these coefficients should be close to zero. However, existence of k coefficients sufficiently large (e.g., larger than 0.5), where k<d, indicate a sufficiently close k-dimensional subspaces of human-selected and NN features. In order to reliably assess the amount of shared information between the two feature spaces, the study compared the number of pairs with a high correlation coefficient discovered by CCA to the reduced number of features obtained by principal component analysis (PCA) [22] that explained most of the variance.

Extraction of selected human features from neural network features. The study represented single human-selected features as linear combinations of NN features. The study identified the i^{th} training and testing student model human selected features with the i^{th} rows of the matrices Zₜᵣₐᵢₙ and Zₜₑₛₜ, which the study denote by zᵢ^{train} and zᵢ^{test} respectively. The study linearly regressed zᵢ^{train} against the rows of Xₜᵣₐᵢₙ. That is, the study found a 245×1 vector wᵢ and a real number b and fit a standard least-squares linear regression model zᵢ^{train}=Xₜᵣₐᵢₙ wᵢ+b1_{N×1}, where 1_{N×1} is an N×1 vector of ones. The corresponding R² statistic, which incorporated the testing data, was interpreted as the linear explainability score. It has values between 0 and 1, where 1 designates perfect linear explanation and 0 an irrelevant vocabulary for linear explanation. It is computed as follows

$${R}^{2} = 1 - {‖ {z}_{i}^{test} - ( {X}_{train} w + b {1}_{N \times 1} ) ‖}^{2} / {‖ {z}_{i}^{test} - \overline{{z}_{i}^{test}} {1}_{N \times 1} ‖}^{2} .$$

For human-selected features that were extracted from each of the leads (for example: T amplitude), the study also tested the ability to reconstruct the averaged feature value across leads.

To verify that the network ability to reproduce the human features is not derived from a simple correlation between the human selected features and the patient age and sex the study calculated the following: the correlation of each human selected feature with patient age and sex as well as the area under curve (AUC) for detecting the patient's sex using that single feature alone.

Results. Using human features in a student model to explain neural network output (FIG. 2B).

The study predicted the output of the two neural networks (age and sex) using human features via linear and nonlinear student models. The study quantified the variance information explained by these models via their R² statistic. For example, R² of value 1 means that the study can explain 100% of the neural network outputs using human features. For age estimation, the linear student model explained 57.1% of the variance (R²=0.571). A nonlinear student NN with two layers explained 70.2% of the variance (R²=0.702). The difference between the two (13.1%) is evidence of the nonlinear use of these features by the deep neural network. In fact, the NN uses a similar nonlinear model after its convolutional blocks.

For sex classification, the linear student model explained 49.4% of the variance (R²=0.494). The nonlinear student model explained 68.5% of the variance (R²=0.685), where the difference between the nonlinear and linear explainability (19.3%) was even greater. Indeed, a linear model is often less useful for a binary classification than continuous regression.

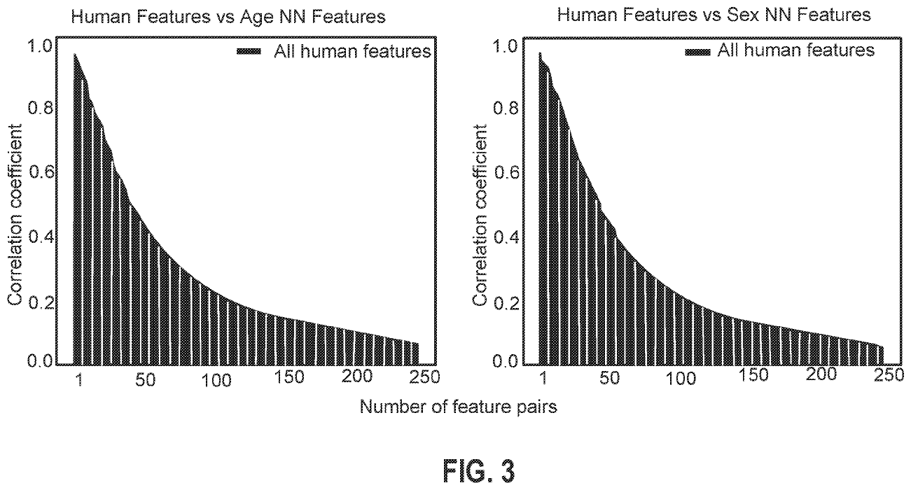

Using canonical correlation analysis to assess the overall correlation between the feature spaces (FIG. 2C). The canonical correlation coefficients for both sex classification and age estimation is shown in FIG. 3. In the age model, 13 of the 245 feature pairs had canonical correlation coefficients of 0.85 or higher and 8 of those had a coefficient of 0.9 or higher. For the sex model, 15 of 245 of the feature pairs had canonical correlation coefficients of 0.85 or higher and 10 of those had coefficients of 0.9 or higher.

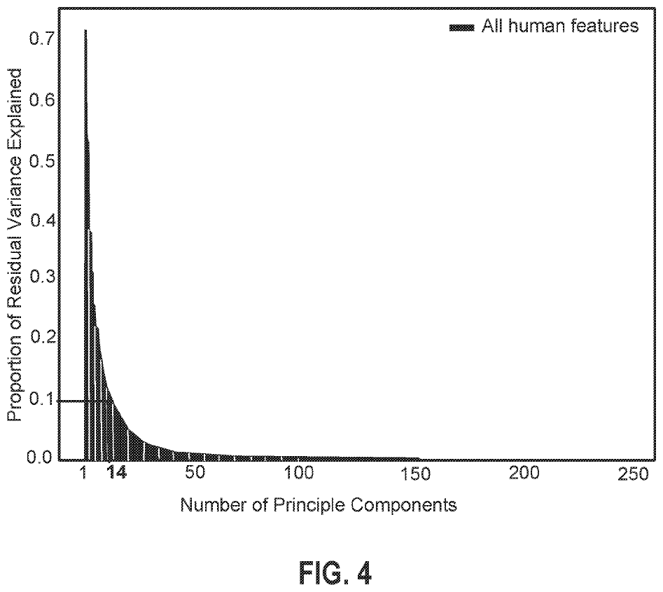

While 13 and 15 out of 245 may seem like a small number of pairs, it is important to note that human-selected features are linearly correlated to one another due to biological reasons. Indeed, FIG. 4 depicts the proportion of residual variance explained as a function of principal components. It emphasizes that the first 14 principal components explain 90% of the human feature variance (see red lines in this figure).

Human features extraction from the neural network features (FIG. 2D)). To further understand the relationship between the two kinds of features, the study created linear models to reconstruct human-selected features from neural network features. Table 1 reports the R² statistic as a measure of variance explainability for human features in the two networks (sex or age). If the feature is computed for each lead separately, and not derived from all 12 leads, then the table reports the maximal value of the R² statistic from all leads and the R² statistic of the average feature value across leads. A supplementary material table further presents the R² statistic of all features including all leads, and more importantly, the R² statistic between each human selected feature and patient age or sex as well as the AUC for detecting the patient's sex using that single feature alone. The feature with the highest correlation and AUC was “Maximum R Amplitude”; it R² static of 0.13 for age estimation and AUC of 0.68 for detection of sex.

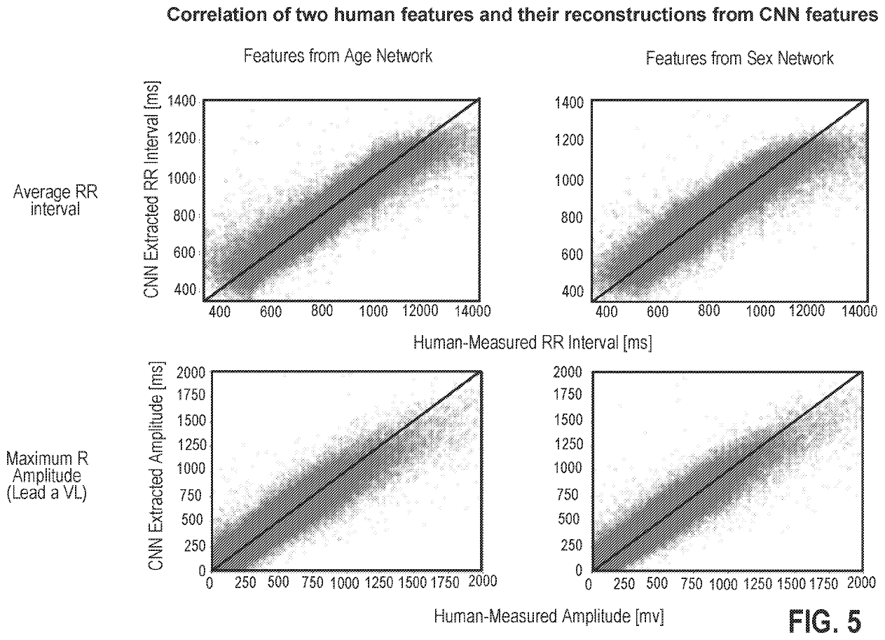

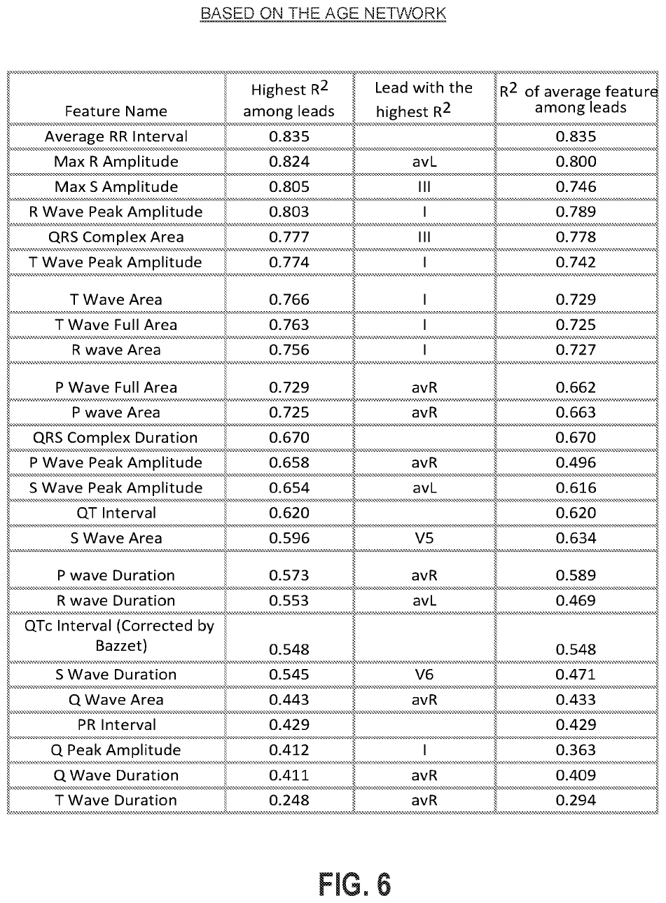

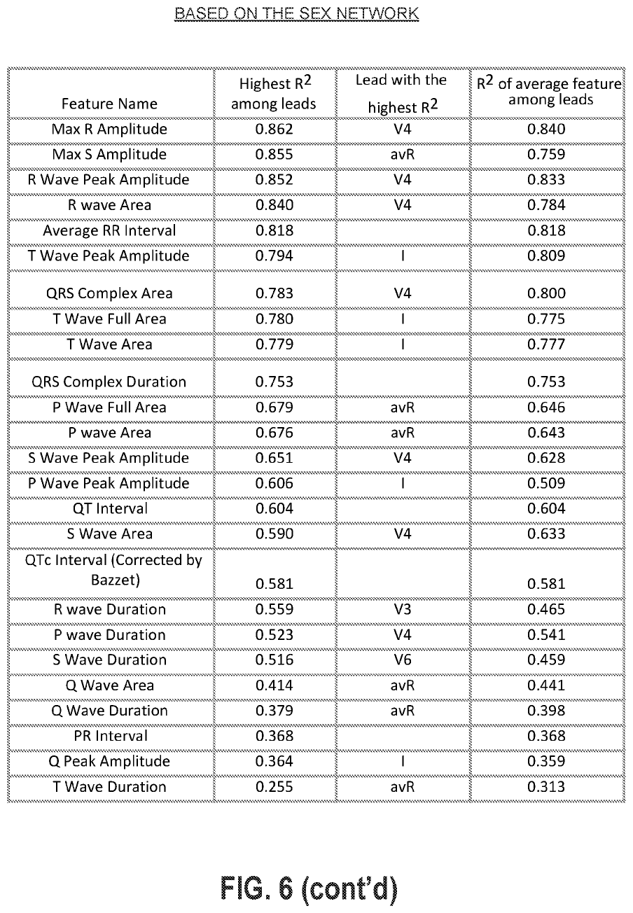

FIG. 5 demonstrates the strong correlation between each feature value (depicted on the x axis) and its reconstruction from the NN using the linear regression model (depicted on its y axis) for two features (average RR Interval and maximal R amplitude) in both networks. Interestingly, for the age network, the feature with the highest R² was the patient heart rate (average RR interval) even though there is practically no correlation between the patients age in our study to their heart rate (R²<0.001). In addition even though the age and sex networks were trained separately, each with a different objective, and had different NN features spaces, when extracting the human-selected features from the two different NN feature spaces, in both cases the same set of features had high R² values.

Discussion. In this work the study sought to determine whether the features selected by neural networks designed for ECG analysis are human understandable features. The study also sought to assess whether the difference between the classification capabilities of neural networks and humans stem from the use of different signal features, the nonlinear nature of neural networks, or both. The study summarize our findings as follows: 1. Neural networks for ECG signals predominantly use features that are correlated with human understandable features; 2. Human selected features, however, explain only part of the neural network model output. For sex classification the study found a 70.2% variance explanation with a nonlinear model and for age estimation it was 68.5%. Thus, identification of novel features (signal components not part of the current vocabulary used to describe ECG signals) by the network seems to contribute to the superior performance of neural networks; The nonlinear nature of neural networks also contributes to their superior performance. Indeed, the linear student models for both age estimation and sex classification were able to explain less than the nonlinear student models.

In summary, neural networks predominantly use human-recognizable features, but then add additional non-human labelled features and nonlinearity, accounting for their superior performance compared to traditional methods. Additionally, as the NN features were extracted without any specific feature engineering, errors in human feature creation may be eliminated and extraction time significantly shortened, as it does not involve manual review of each tracing.

The demonstrated ability to derive known ECG features with biological meaning from NN features in a linear way may means that these features are not unique to human intelligence. Indeed, two different neural networks (age and sex classifiers) seem to utilize the same human-selected features without any a-priori knowledge of what an ECG signal should look like, including the detection of features that are uncorrelated with the model labels. For example, the age estimation model demonstrated strong ability to estimate the ECG heart rate from the neural network features (R²=0.835) with almost no correlation between the patient age and their heart rate (R²=0.0009). Not all human-identified features were used by the neural networks. This might be considered a limitation, but the study believe this it is another sign that each network underwent a meaningful learning process resulting in the selection of features that have a direct association with the classification task it was assigned.

Furthermore, the study were not able to perfectly explain the output of the model using the vocabulary of human-selected features, that is, the R² score was less than 1. There are three potential explanations for this finding. The first is that the neural network found features that reflect components of the signals not defined by most humans, including features that are often described as “gestalt”, these almost invisible signs that appear to an expert physicians might be hard to explain using any natural language and hard coded rules. The second is that the vocabulary used by humans to describe signal features is somehow ambiguous and the definitions of some feature elements lack sufficient accuracy to provide robust classification. The last is that the network found false associations, for example, a feature that was present in the training set but was not generalizable or relevant for common instances. Such features represent a bias in the training set and might be exploited to permit a simple adversarial attack. To improve explainability in such cases one may apply adversarial training and possibly noise injection. This might happen when one fools a neural network with an insignificant change in the signal that would not affect human classification (the human may not even see it), but that would lead the network to misclassify the tracing.

While our work is focused on ECG analysis, and ECG-based features, the study present a general framework to extract and compare neural network features and human-selected features. In particular, the study suggest student models and simple quantitative methods of correlating and explaining human-selected features using neural network features. The study thus expect our methods to apply to other fields, where human-engineered features exist.

FIG. 2 depicts various classifier architectures configured to use human-selected or neural-network selected features. Panel A: The top of this panel shows human-based classifiers use expert selected features, and apply a model to create a classification (e.g. ST segment elevation to classify MI); the bottom of this panel shows a neural network uses convolutional layers to extract signal features, and then feeds those inscrutable features into the model (in the examples here, fully connected layers). Panel B: Use of human-selected features in a student model to predict neural network output. The extent to which the student model predicts neural network output is indicative of the extent to which human-selected features may be used by the neural network. Panel C: Canonical correlation to assess the overall correlation between human-selection features and the features selected by the convolutional layers (feature extraction layers) of the neural network. Panel D: Use of a linear model to reconstruct human-selected features from neural network selected features, to further assess their relationship.

FIG. 3 depict plots demonstrating canonical correlation between human-selected and neural network-selected features. The canonical correlation analysis describes the correlation between the human-selected features and the age estimation neural network selected features (left) and between the human-selected and neural network-selected features of the sex classification network (right). Each bar represents the correlation coefficient between one pair of features from both spaces (neural network feature space and human-selected feature space) after they have been de-correlated, in a process similar to principal component analysis.

FIG. 4 depicts a plot demonstrating a proportion of residual variance explained as a function of principal components. The proportion of explained variance in the human explainable feature space. Since the features have inherent biological correlations, the study used principal component analysis to quantify the number of unique features. As seen in the figure, 14 features explain 90% of the information in the human-selected feature space.

FIG. 5 depicts an estimate of human-selection features using neural network-selected features. Two examples of two human-selected features that were reconstructed in a linear manner from the neural network feature space, age estimation network features (left) and sex classification network features (right). Even though the networks were trained separately, both networks possess a similar ability to reconstruct specific human identifiable features, which are nonlinear in nature (average RR interval in the upper panels and maximum R-wave amplitude in the lower panels).

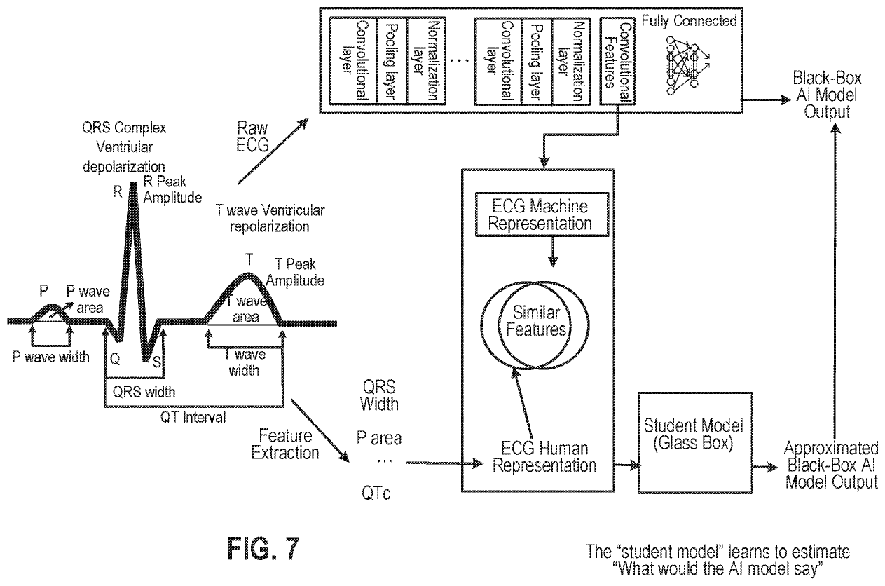

Explaining the features selected by a neural network or other machine-learning model becomes an important task to discover new relations between diseases and ECG patterns, thereby creating new medical knowledge. Two pathways for solving this problem include (1) synthesizing ECGs using GANs as described in further detail in the following section, and (2) the master-student structure as shown in FIG. 7. As to the master-student structure, the study will train a shallow and explainable network called the “Student network” (for example a logistic regression with handcrafted features as inputs) to perform a task that was done by the deep convolutional neural network—the “Master network”—for example Detection of low EF. While using the features to predict the hard outcome (0/1) yields a low accuracy model, when training the model on the output of the master network, the accuracy improves, and it seems as the master network is used to curate and clean the labels. The “Master network” not only improves the accuracy of the student network, but also allow us to understand how the explainable features effected the master network decision-practically explaining the features that drives it.

### Example 2—Synthesizing Physiologically Biased ECGs Using Modified GAN

Generative adversarial networks (GANs) ca be used to reverse engineer black box models and create synthetic datasets that visually and interactively demonstrate feature changes as the network probability is adjusted. GANs include a discriminator (D) and generator (G). The discriminator is designed to classify inputs as “real” or “fake” (synthetic) and the generator aims to fool the discriminator. During training the discriminator is presented with real samples, whereas the generator is fed noise that it can use as a seed to create synthetic samples. As the generator is deterministic, a practical way to prompt creation of a variety of synthetic samples, is to feed in a random seed, which will be translated by the generator to a synthetic sample. Using discriminator scores and gradients, the generator progressively creates more realistic synthetic data, until the discriminator can no longer differentiate synthetic from real data.

An expert model can be added to the GAN architecture. The expert model serves as the target for explanation, and a GAN as the tool to reverse engineer the expert model. The generator receives gradients from the discriminator and from the expert and tries to generate an ECG that is both realistic (so as to be accepted by the discriminator) and with a specific continuous label (evaluated by the expert model).

In some implementations, a generator is trained to reverse engineer an ECG expert model, e.g., an AI-ECG sex model, so that for any input probability in the range [0, 1] indicating a likelihood of being male or female, and a random seed, a synthetic ECG can be generated that is biased to include features tuned to the input probability.

The loss function for the generator can include a component that rewards the generator for fooling the discriminator and also a component to minimize the difference between the requested output and the expert score. In some implementations, the mean absolute error function optimizes both the score and the appearance of the ECG, so that it looks real to a human expert, even with mid range labels (0.5 probability of being a male). To allow the use of non-binary model outputs, which will make the term absolute value bigger if the expert model outputs are not limited to a number between 0 and 1, a scaling factor alpha can be added.

Given a convolutional neural network, h(x)→[a, b], where x is an ECG and h(x) is the model output (for example, in the sex model a=0, b=1 and the output is the probability of being male), h(x) can be explained by finding all the inputs x that h(x)=y. For this purpose, we look for h⁻¹(y)=x, y E [a, b], even though h(x) is not invertible. A pseudo-inverse h_{z}⁺(y)=x_{z} of h is a function satisfying h(h_{z}⁺(y))=y. The pseudo-inverses can be parameterized by z. This way, given z, a sample can be found that will yield the wanted y if fed to the original network h(x).

The two practical requirements from the pseudo-inverse are: h_{z}⁺(y) looks realistic, i.e. looks as it was sampled from the original space of x_{z}, h (h_{z}⁺(y))=y. An important limitation in some cases is that if h(x)=y, and a sample x_{z} is generated by using h_{z}⁺(y), the generated x_{z} will be almost always be different from x, and, since adversarial training is used to create h_{z}⁺(y), it cannot be guaranteed that x_{z}=x as it isn't known if any ECG can be generated or just a partial set of ECGs.

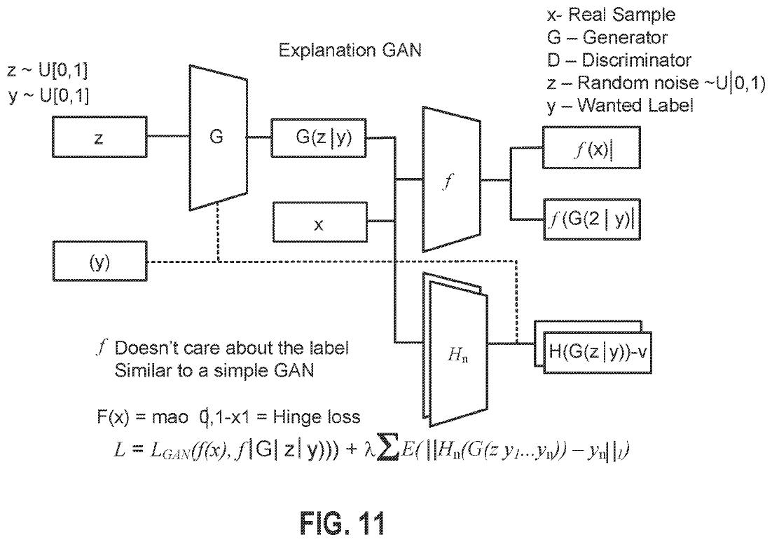

As seen in FIG. 11, the generator output is evaluated by both the expert model and the discriminator. The discriminator optimizes the generator to create realistic samples and is governed by the adversarial loss. By creating only realistic samples, the first requirement of the pseudo-inverse is satisfied. The expert model, on the other hand, assumes that the inputs are from the same distribution as the real inputs, and uses the generated sample to calculate the labels. By using the gradients from the expert network, the generator is trained to minimize the second term of the loss, ∥h(G(z,y))−y∥₁. Furthermore, for an optimal generator, h(g(y))=y, satisfying the second requirement of the pseudo-inverse (as we want to maintain the balance between the adversarial loss to the expert loss terms), λ can be used, i.e., a hyperparameter to keep the balance between the two requirements of the pseudo-inverse.

As seen in FIG. 11, the generator can be conditioned by expanding its latent space. While some GANs use a completely random latent space based on noise, the present system adds to the noise in a space that is a learned embedding of the label, and when using the same noise, but changing the labels, we receive samples that have some similar properties (the ones encoded by the common latent space) and also some different ones (that contribute to the different label).

A model can also be created that will generate ECGs with more than one condition, by connecting the generator to more than one h(x) and minimizing Σ∥hᵢ(G(z,y))−yᵢ∥₁. The resulting ECGs will yield different outputs from the different expert models.

For example, a system can be implemented that uses both the sex classification expert model and the low EF classification expert model, such that the latent space will encode more than one condition. Since the two models were trained on real ECGs, and the different labels are not necessarily independent (as a male, for example, may have a higher chance of developing low EF, there might be a correlation between the expert model outputs.

When training, the system optimizes both the adversarial loss that enforces that the model output will have similar properties as those of a real ECGs. The expert model terms in the loss function further minimize the difference between the requested label and the label of the generated ECG. In essence, we are creating adversarial attacks, in order to fool the expert models (as the inputs are not real ECGs, and the sex, and ejection fraction is not defined) and force it to output a specific value, while at the same time force the generator to create samples that look realistic. To maintain the quality of the outputs, different loss weights have been applied during training to maintain this balance, and for example, it was found that a scale of 2:1 between the expert loss to the adversarial loss was specifically useful, and that the model converged faster when the ECGs were rescaled by a factor of 2000 (95% had an amplitude of between −1 to 1 after rescaling).

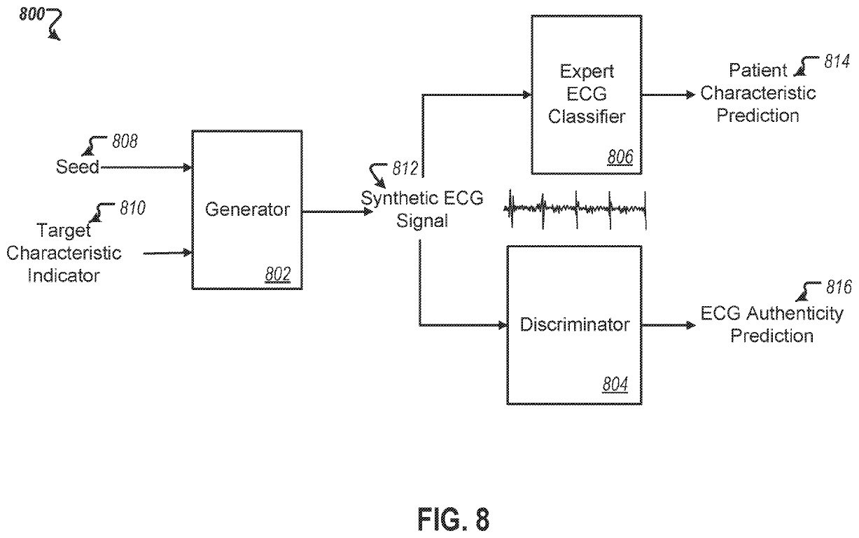

FIG. 8 depicts an explanatory generative adversarial network system 800 for training a generator machine-learning model to produce realistic and physiologically-biased synthetic ECG signals. EGAN 800 includes a generator 802, an expert ECG classifier 806, and a discriminator 804, each of which are machine-learning models such as artificial neural networks. In some implementations, the generator 802 and discriminator 804 are both convolutional neural networks, although other architectures can alternatively be employed. Generator 802 is configured to process as inputs a seed 808 and a target characteristic indicator 810. Seed 808 is a randomly selected value, e.g., determined using a random or pseudo-random function. Target characteristic indicator 810 represents a target physiological characteristic for a patient. The target characteristic indicator 810 is a label that indicates how the synthetic ECG signal 812 should be conditioned/biased to reflect a particular physiological characteristic. The target indicator 810 can be a non-binary value that can assume any value in a defined range of values. For example, a value between 0 and 1 can selected as the target characteristic indicator 810, where a value of 0 requests that the synthetic ECG signal be generated to include features that are strongly correlated with a female; a value of 1 requests that the synthetic ECG signal be generated to include features that are strongly correlated with a male; and values between 0 and 1 are more or less strongly correlated with female or male ECGs. In some implementations, the physiological characteristic represented by the target characteristic indicator 810 is a sex of the patient, an age of the patient, or a ventricular function of the patient (e.g., an ejection fraction characteristic, a heart rate, an arrhythmia, or a left ventricular dysfunction).

The generator 102 is trained to create a synthetic ECG signal 812 that is biased/conditioned based on the target characteristic indicator 810. The ECG signal 812 can describe a full 12-lead ECG signal, a single lead ECG signal, or any other number of appropriate leads. Notably, the invention is not limited to synthesizing ECG signals. The techniques disclosed herein can also be applied to synthesize electroencephalogram (EEG) signals, and other electro-biological signals, for example.

Expert ECG classifier 806 is a machine-learning model trained to generate predictions regarding a patient characteristic based on processing inputs representative of an ECG of a patient. The expert classifier 806 may process an ECG signal directly, features derived from the ECG signal, or both. The predicted patient characteristic 814 corresponds to the same physiological characteristic represented by target characteristic indicator 810, e.g., a sex of the patient, an age of the patient, or a ventricular function of the patient (e.g., an ejection fraction characteristic, a heart rate, an arrhythmia, or a left ventricular dysfunction). The patient characteristic 814 can be a percentage or other value that indicates a likelihood that the patient exhibits the specified physiological characteristic (e.g., that the patient is a male, that the patient is a female, that the patient has left ventricular dysfunction).

Discriminator 804 is trained to distinguish authentic from inauthentic ECG signals, and processes an input representing an ECG signal of unknown type to generate an ECG authenticity prediction 816 that indicates whether or a likelihood that the inputted ECG signal is real (authentic) or not (inauthentic). During training, the discriminator 804 the inauthentic inputs can be inputs that do not represent ECG signals at all, and/or can be synthetic ECG signals, e.g., synthetic ECG signal 812.

Although just one expert classifier 806 is depicted in FIG. 8, some implementations can include multiple expert classifiers. In such cases, the generator 802 can be trained to condition or bias the synthetic ECG signal 812 based on multiple target characteristic indicators 810 corresponding to different physiological characteristics.

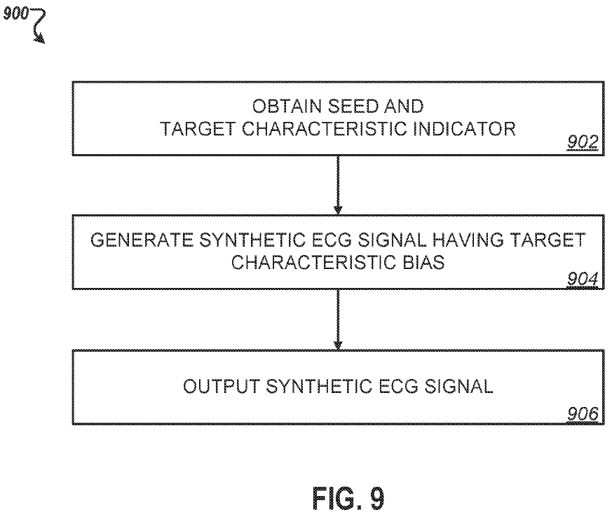

FIG. 9 depicts a flowchart of an example process 900 for generating a physiologically biased synthetic ECG signal. The process 900 obtains a seed and a target characteristic indicator (902). A generator machine-learning model processes the seed and the target characteristic indicator to generate a synthetic ECG signal biased/conditioned according to the target characteristic indicator (904). The synthetic ECG signal can be stored, transmitted, and or displayed, and can include visually recognizable features that correspond to the physiological characteristic represented by the target characteristic indicator (906).

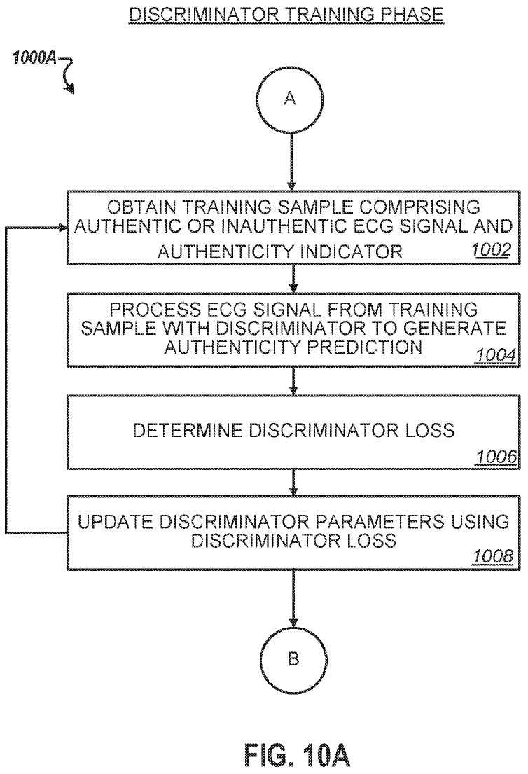

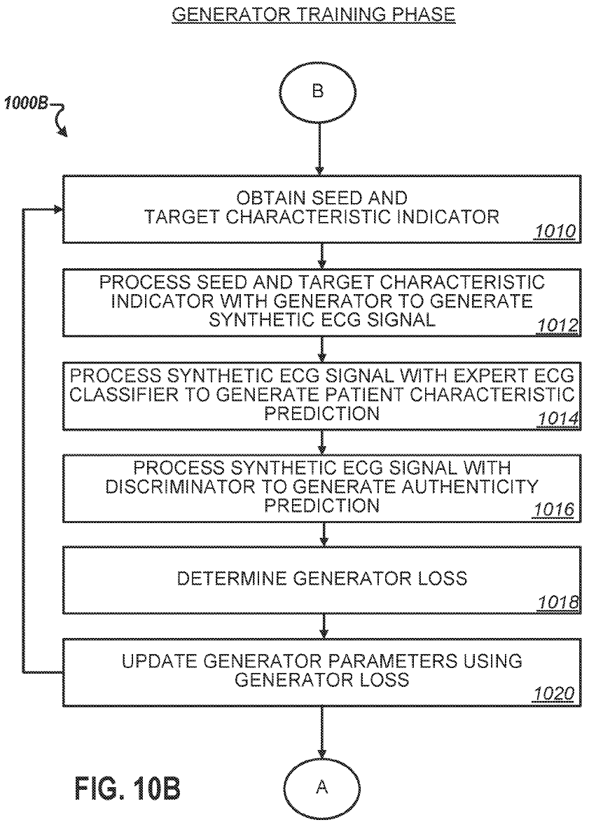

FIGS. 10A-10B depict flowcharts of an example process 1000 for training an explanatory generative adversarial network for physiologically-biased ECG signal creation. In general, process 1000 proceeds in two phases, i.e. a discriminator training phase 1000A (FIG. 10A), and a generator training phase 1000B (FIG. 10B). The training procedure can alternate iteratively between the two phases, where discriminator parameters are updated during the discriminator training phase (and generator parameters are held constant), and where generator parameters are updated during the generator training phase (and discriminator parameters are held constant).

During the discriminator training phase, the process 1000A obtains a training sample comprising an authentic or inauthentic ECG signal and an authenticity indicator/label that indicates whether the ECG signal is or is not authentic (1002). The discriminator processes the ECG signal from the training sample to generate an authenticity prediction (1004). A discriminator loss is determined (1006), where the discriminator loss is based on a difference or other comparison between the authenticity indicator/label and the authenticity prediction. The system then trains the discriminator by updating trainable parameters of the discriminator using the discriminator loss (1008). For example, the discriminator can be a neural network having trainable weights, which are updated by back-propagating the discriminator loss through the discriminator, computing a gradient, and updating the weights accordingly.

During the generator training phase, the process 1000B obtains a seed and a target characteristic indicator (1010). A generator machine-learning model processes the seed and the target characteristic indicator to generate a synthetic ECG signal (1012). The expert ECG classifier processes the synthetic ECG signal to generate a patient characteristic prediction (1014), and the discriminator processes the synthetic ECG signal to generate an authenticity prediction (1016). The system determines a generator loss (1018), where the generator loss can include two components: an authenticity component and a patient characteristic component. The authenticity component is based on the authenticity prediction, where a larger loss is indicated for the generator when the authenticity prediction more confidently predicts that the synthetic ECG signal is inauthentic, and a smaller loss is indicated for the generator when the authenticity prediction more confidently predicts that the synthetic ECG signal is authentic. The patient characteristic component is based on the patient characteristic prediction, and can indicate an error/difference between the target characteristic indicator and the patient characteristic prediction. The patient characteristic component of the loss typically increases as the difference between the target characteristic indicator and the patient characteristic prediction increases, and the patient characteristic component of the loss typically decreases as the difference between the target characteristic indicator and the patient characteristic prediction decreases. Backpropagation is used to determine gradients for the discriminator and the expert classifier, and the error is propagated through the generator to update the trainable parameters (e.g., weights) of the generator model to as to optimize the loss function, e.g., by minimizing the patient characteristic and authenticity components of the generator loss (1020).

### Computer-Based Implementations

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A computer-implemented method, comprising:
obtaining first values for a first set of features from a first machine-learning model, wherein the first values for the first set of features were determined through a process of training the first machine-learning model to perform a particular classification task based on inputs that represent a signal;
obtaining second values for a second set of features from a second machine-learning model, wherein the second values for the second set of features were determined through a process of training the second machine-learning model to perform the particular classification task based on inputs that represent morphological features of the signal;
processing the first values and the second values to correlate at least a subset of the first set of features with at least a subset of the second set of features; and
using the correlation to update the first machine-learning model, update the second machine-learning model, or train another machine-learning model.

2. The method of claim 1, wherein the first machine-learning model and the second machine-learning models are neural networks.

3. The method of claim 1, wherein the signal is an electrocardiogram (ECG) or an electroencephalogram (EEG).

4. The method of claim 1, wherein the morphological features of the signal are human-selected features, wherein the first set of features includes features are not human-selected features.

5. The method of claim 1, wherein the first set of features correspond to a last hidden layer of a neural network.

6. The method of claim 1, comprising using the correlation to update the second machine-learning model by reducing the second set of features, or using the correlation to update the first machine-learning model by reducing the first set of features.

7-8. (canceled)

9. A method for training a computer-implemented system to generate synthetic electrocardiogram (ECG) signals, comprising:
obtaining a seed and a target characteristic indicator, wherein the target characteristic indicator represents a target physiological characteristic for a patient;
processing, with a generator machine-learning model, the seed and the target characteristic indicator to generate a synthetic ECG signal;
processing, with an expert machine-learning model, the synthetic ECG signal to generate a patient characteristic prediction;
processing, with a discriminator machine-learning model, the synthetic ECG signal to generate an authenticity prediction;
determining a generator loss based on (i) a first comparison of the patient characteristic prediction to the target characteristic indicator and (ii) a second comparison of the authenticity prediction an authenticity indicator that indicates the synthetic ECG signal was inauthentic; and
updating parameters of the generator machine-learning model based on the generator loss.

10. The method of claim 9, wherein the target physiological characteristic of the patient is a sex of the patient, an age of the patient, or a ventricular function of the patient.

11. The method of claim 10, wherein the ventricular function of the patient comprises an ejection fraction, a heart rate, an arrhythmia, or a left ventricular dysfunction.

12. The method of claim 9, wherein the target characteristic indicator represents the target physiological characteristic for the patient on a continuous, non-binary scale.

13. The method of claim 9, wherein the expert machine-learning model is pre-trained to generate patient characteristic predictions based on ECG signals, the patient characteristic prediction comprising sex, age, or ventricular function.

14. The method of claim 9, wherein the seed is a randomly selected value within a range of values.

15. The method of claim 9, wherein the generator machine-learning model comprises a first convolutional neural network and the discriminator machine-learning model comprises a second convolutional neural network.

16. The method of claim 9, comprising alternately training the generator machine-learning model and the discriminator machine-learning model, wherein parameters of the discriminator machine-learning model are held constant while training the generator machine-learning model, wherein the parameters of the generator machine-learning model are held constant while training the discriminator machine-learning model.

17. The method of claim 9, wherein updating the parameters of the generator machine-learning model based on the generator loss comprises back-propagating the generator loss through the discriminator machine-learning model, the expert machine-learning model, and the generator machine-learning model, and using gradients from the back-propagation to update the parameters of the generator machine-learning model.

18. The method of claim 9, wherein determining the generator loss comprises weighting the first comparison (an expert loss) greater than the second comparison (an adversarial loss).

19-20. (canceled)

21. A method for generating a synthetic electrocardiogram (ECG) signal, comprising:
obtaining a seed and a target characteristic indicator, wherein the target characteristic indicator represents a target physiological characteristic for a patient; and
processing, with a generator machine-learning model, the seed and the target characteristic indicator to generate the synthetic ECG signal, wherein the generator machine-learning model biases the synthetic ECG signal according to the target physiological characteristic represented by the target characteristic indicator.

22. The method of claim 21, wherein the target physiological characteristic of the patient is a sex of the patient, an age of the patient, or a ventricular function of the patient.

23. The method of claim 22, wherein the ventricular function of the patient comprises an ejection fraction, a heart rate, an arrhythmia, or a left ventricular dysfunction.

24. The method of claim 21, wherein the target characteristic indicator represents the target physiological characteristic for the patient on a continuous, non-binary scale.

25-29. (canceled)
